(19) United States
(12) Patent Application Publication
(10) Pub. No.: US 2013/0030272 A1
(43) Pub. Date: Jan. 31, 2013

(54) TONOMETRIC DEVICE FOR EXAMINING RESPIRATORY INSUFFICIENCY AND REGIONAL TISSUE PERFUSION FAILURE

(71) Applicant: Domokos Boda, Szeged (HU)

(72) Inventor: Domokos Boda, Szeged (HU)

(21) Appl. No.: 13/521,336

(22) PCT Filed: Sep. 1, 2010
(86) PCT No.: PCT/HU10/00093

§ 371 Date: Jul. 25, 2012

(30) Foreign Application Priority Data
Sep. 1, 2009 (HU) P0900537

(51) Int. Cl. A61B 5/145

(52) U.S. Cl. 600/363

(57) ABSTRACT

A probe comprising a tubular member arranged substantially in a plane within a perimeter fitting into the sublingual or postlabial area of a human patient. The tubular member is made preferably of silicone rubber or a material permeable for gases, particularly for carbon dioxide, and forming a barrier against body fluids. The tubular member has consecutive curved sections communicating with each other and form a continuous flow pass. Ends of the tubular member have connection tubes adapted for delivering the fluid inside the lumen of the tubular member directly or indirectly to a measuring apparatus. The connection tubes are made preferably of a material substantially impermeable for carbon dioxide.

The present invention relates to an improved tonometric device for examining respiratory insufficiency and regional perfusion failure of the body.

The tonometric devices have been widely used in the medical practice for the examination of the tissue-specific partial pressure belonging to the carbon dioxide concentration in the whole gastrointestinal tract, especially in the stomach, the partial pressure of carbon dioxide being referred to as pCO₂, particularly as pgCO₂ in the case of measuring inside the stomach and/or the gastrointestinal system. In steady-state circular conditions this value is almost equal to that of the arterial blood; therefore the method proved to be useful for detecting the failures of the respiratory regulatory system. On the contrary, in the case of patients having acute conditions because of various origins the tissue-specific pCO₂ value is higher than that in the blood. The difference between the two values is referred to as pCO₂ gap. By examination of this gap the regional tissue perfusion failure can be detected, and from its size the severity of the particular disorder and the extent of the endangeredness of the patient can be concluded.

The experience acquired with numerous patients has shown that on the one hand tonometry is useful for controlling the respiratory performance by determining the systemic partial pressure related to carbon-dioxide concentration, and on the other hand this is the easiest, cheapest and most reliable method for predicting the hazard of acute disorders in patients and for monitoring the conditions of patients.

Patent specification U.S. Pat. No. 4,463,192 discloses a solution for tonometric measurements. The device disclosed therein is a probe provided with a balloon, which is readily permeable for gases but impermeable for other substances and this probe is introduced into the stomach, whereafter the balloon is filled with air or a liquid from outside. After a while, the substance filled into the balloon takes up by diffusion the value of the carbon-dioxide concentration of the blood circulating in the gastric wall (mucous membrane) and this value can be determined by relatively simple laboratory methods.

The above-mentioned commonly used probe and examination method have several disadvantages. The introduction of hard and thick probes into the stomach causes discomfort to the patient, thus the method is essentially invasive. The saturation time of the relatively high amount of filling liquid with carbon dioxide inside the balloon is too long, and thereafter the result must still be corrected for calculating the final data.

Patent specification U.S. Pat. No. 6,216,023 discloses a more preferable solution wherein the balloon is loaded with air, and the obtained sample is analyzed with a gas analyzer (capnometer). New devices are applied for the automatic sampling of the air and the simple transport of the air by recirculation. Nevertheless, a probe supplied with a balloon is used for this method whereby it is also aggressive, and for obtaining reliable results the patients may need to be pre-treated with drugs.

For examination of tissue perfusion failure with tonometric methods electrochemical and fibre-optic sensors have also been used. Patent specifications U.S. Pat. No. 6,216,024, and U.S. Pat. No. 6,143,150 disclose such solutions. According to this methodology the sensor itself is introduced into the stomach of the patient by using a probe. Sometimes the measurement is performed with a sensor arranged in the upper digestive tract, instead of the stomach, but the examination method still involves discomfort and is considered as invasive.

Patent specification U.S. Pat. No. 5,423,320 discloses a tonometric method and apparatus for measuring and monitoring pCO₂ in the stomach and the intestines. By using air. For this purpose a CO₂ sensor, optionally a pO₂ sensor is used, and in situ measurements are carried out at the end of the sensor, or a specified amount of air is circulated through a catheter the distal end of which is introduced into the cavity of the patient to be examined. The catheter includes a detector, at one side of which a gas permeable membrane is arranged. The CO₂ sensor of the apparatus is fixed to the catheter and provides a signal characteristic to the partial pressure of CO₂.

International publication WO 2005/041764 discloses a tonometric device designed substantially as a tube that does not comprise any balloon, but has a tube section to be introduced into the gastrointestinal tract of the patient, said section made of a material permeable for gases, especially for carbon dioxide, but substantially impermeable for other substances of body fluids

Tonometric devices outlined above have to be introduced into the stomach of the patient. Steadily effected examinations using devices steadily inserted into the stomach are carried out to track and monitor the condition patients affected with some serious or critical disease existing in all segments of clinical care. There is a growing demand for tonometric examinations that can be carried out in case patients suffering from some chronic disease lasting for months, and in such cases the examination were encumbered by the probe laying in the stomach for a long period, on one hand, and the examination should be carried out only occasionally, once a day or even seldom, on the other. A further demand consists in that the examination should be appropriate for easy implementation by the patients who are usually wakeful and even without the help of medical personnel. Such clinical aspects comprise (neuromuscular) diseases involving chronic progressive muscular atrophy, circulatory diseases caused by apnoea during dormancy, respiratory disorders caused by excessive adiposity (patients with Pikwick syndrome), panic disorder, particularly obstructive airway disease (Chronic obstructive pulmonary disease, COPD) of elderly people getting nowadays an endemic disease, as well as pulmonary diseases, chronic circulation insufficiency and other diseases referred to as chronic sleep disorders in the literature. A precondition of the success of recent and ever more efficient treatments is just to provide non invasive, simple and cheap means for regularly determining the pCO₂ value of the organism. However, this has been possible by using methods involving taking blood samples.

To meet these demands tonometric measurements carried out in the oral cavity may be suitable. This possibility is mentioned in the literature. Patent specification U.S. Pat. No. 6,055,447 teaches the oral determination of pCO₂. This technique uses a CO₂ sensor to be placed under the tongue of the patient. However, a serious deficiency of the method consists in that the sensors have often to be calibrated between measurements, i.e. they serve virtually for single use, and relevant display units cause substantial additional costs.

Despite of all advantages spreading of oral tonometric tests is hindered so far by the fact that techniques available for this purpose are not sufficiently simple on one hand, and are costly on the other.

The object of the present invention is to provide a tonometric device by obviating the drawbacks of prior methods and apparatuses that is not invasive, i.e. does not require the introduction of the examination device into the oesophagus or stomach, has a higher area/volume ratio, allows easier and cheaper examination of respiratory insufficiency and regional perfusion failure of the body in the oral cavity.

The invention is based on the recognition that the above aim can be attained by using a specially folded silicon rubber tube allowing sublingual (under the tongue) or postlabial (behind the lip) measurement of CO₂ concentration inside the oral cavity of a patient.

Based on the above, the present invention provides a device (probe) for examination of respiratory insufficiency and regional tissue perfusion failure in patients that is characterised in that said device includes a silicone rubber tube comprising adjacent rounded V-shaped sections arranged alongside and connected to each other in a plane within an area having a curvilinear border, said device including further a fibre arranged inside said tube, said tube is provided with two terminals , and said device is adapted to be accommodated in sublingual or postlabial position in an oral cavity of the patient. The curvilinear border has a wide rounded boomerang shape having a concave part. In a position during use this concave part is capable to accommodate both a vinculum (frenulum linguae) and a bridle of the lower lip (frenulum labii inferioris). In case of sublingual use said device can lay into the bottom of the oral cavity, while in case of postlabial use it can be located between a lower lip and lower gum and teeth.

Preferably, the sections of the silicon rubber tube are connected to each other by gluing. Said gluing is not continuous along at least the curved sections but punctual in order to leave the largest possible surface of the tube free. For providing sufficient retentivity, gluing points are arranged preferably along the border defining said area. Said terminals are positioned centrally in a convex part opposite the middle of said concave part.

A preferred embodiment said two terminals are represented by two separate tubes connected to both ends of said silicon rubber tube and to facilitate handling of the device and inserting it into the mouth it is made of a material harder than said silicon rubber tube, preferably of polyethylene.

The fibre within said silicon rubber tube prevents flattening of the tube to allow unhindered flow of air or liquid comprises a fibre having a diameter ranging from 0.2 to 0.3 mm made preferably of a polyamide thread. Said fibre may be a monofilament as well.

The device of the present invention filled up with air or a liquid. Said liquid comprises preferably sodium chloride, sodium hydrogen carbonate and an indicator suitable for detecting carbon-dioxide concentration. Said indicator comprises expediently phenolic red.

The invention relates further to a kit for examining respiratory insufficiency and regional tissue perfusion failure in patients comprising a tonometric device and a measuring means adapted for direct or indirect detection of carbon dioxide and adapted to be connected to said device.

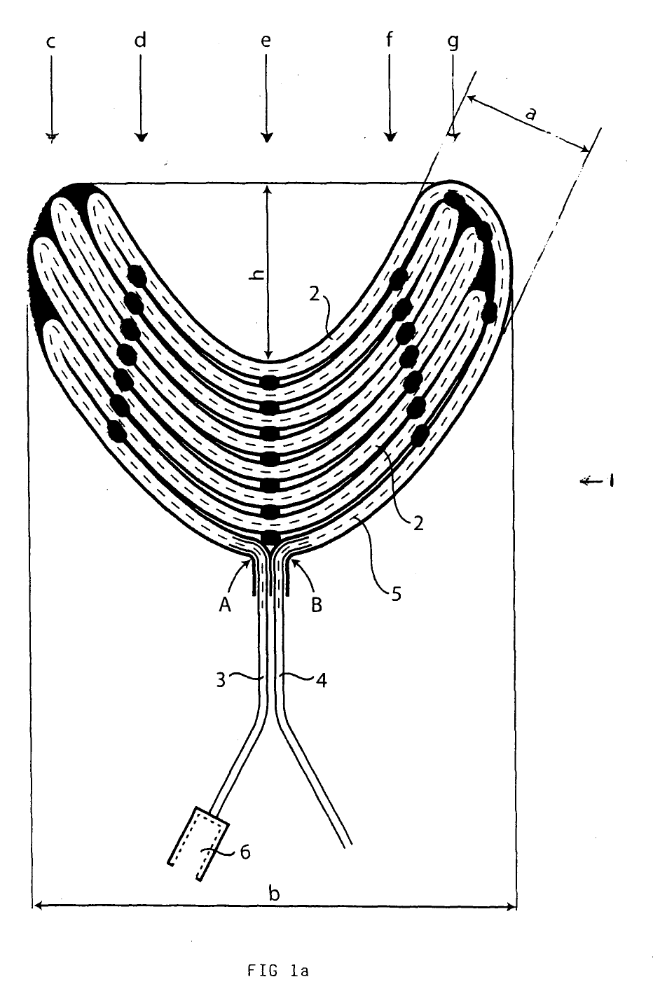

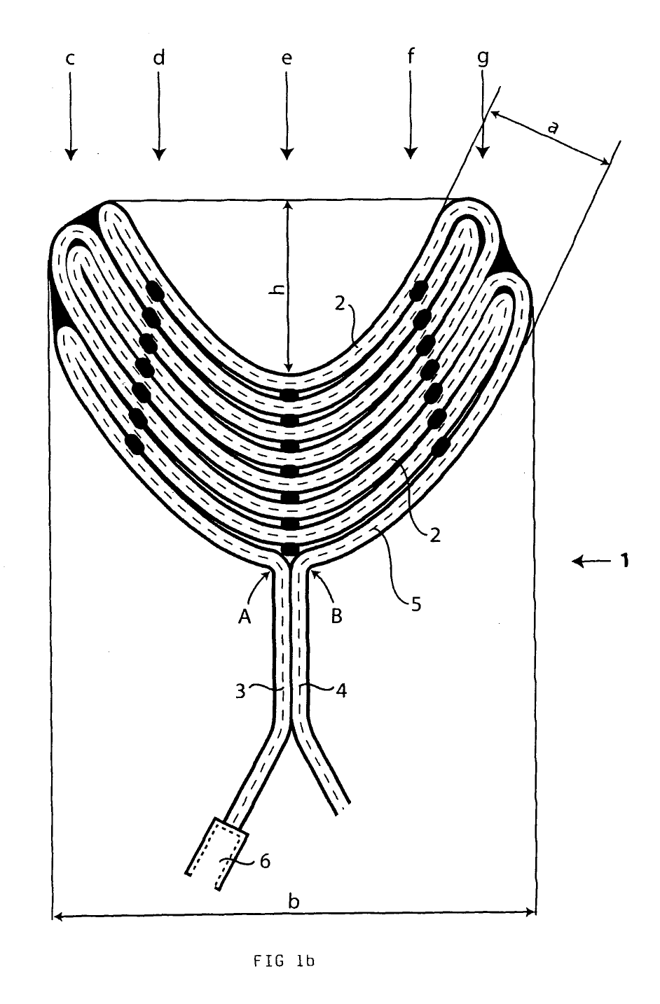

The invention will be described in details with reference to attached FIGS. 1 to 3. In the drawings

FIG. 1a is a plan view of the tonometric device of the invention;

FIG. 1b is a plan view of the tonometric device of the invention; for a case, wherein two terminals are formed by the silicon rubber tube itself, and differing in the folding of the silicon rubber tube from that of FIG. 1a;

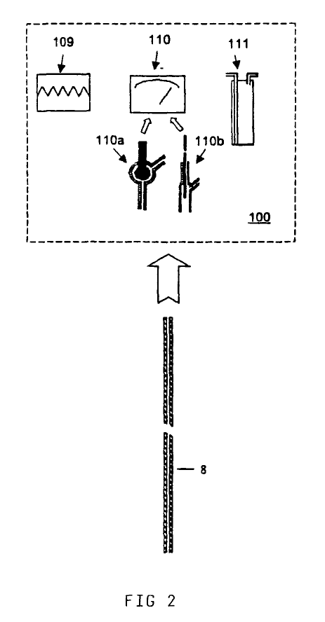

FIG. 2 is a scheme of measuring possibilities performable by the tonometric device of the invention;

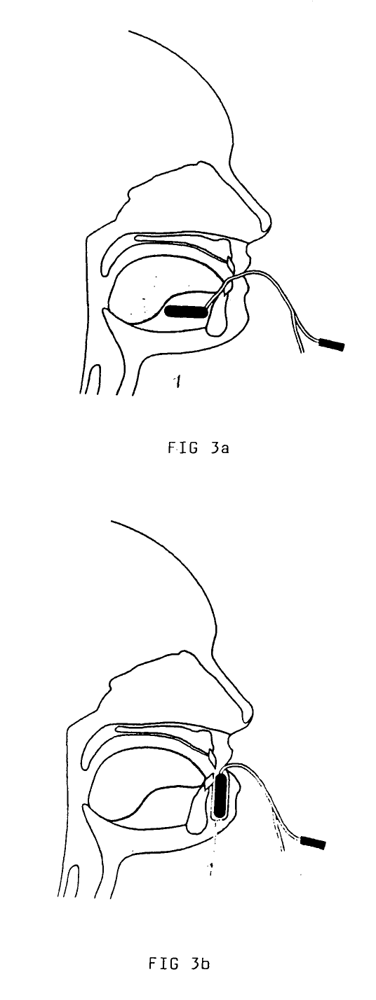

FIGS. 3a and 3b are outlines of sublingual and postlabial placement of the device of the invention.

The definitions of a, b and h is given herewith as follows:

a: is the total width of rounded V shaped sections as measured in the plane of the device;

b: is the distance between ends of rounded V shaped sections

h: is the height of the rounded V shape.

In a preferred embodiment of the device of the present invention a ranges expediently from 16 to 18 mm, b from 16 to 18 mm, h from 19 to 21 mm. The outer diameter of the silicone rubber tube is expediently 2 to 3 mm and has a wall thickness less than 0.3 mm. A tube having an outer diameter of 2.0 mm is preferred.

In a preferred embodiment of the tonometric device 1 of the present invention is prepared from a silicone rubber tube 2 having an outer diameter 2 mm and has a wall thickness of 0.25 mm. The length of the tube 2 is defined to correspond to a volume of 0.8 to 1.2 ml. The tube is folded in a plane to provide several windings to be closely alongside each other as shown in FIG. 1 and is glued along five lines (c, d, e, f, g) such that air or liquid can flow through the tube 2 without any obstruction. For this purpose a fibre made preferably of a polyamide and of 0.2 to 0.3 mm thickness is inserted into the lumen of the tube along its full length, thereby after folding the tube a sufficient clear gap remains to allow test material to be discharged into a measuring unit by drawing. After appropriate folding the starting and final part of the tube are fixed together preferably by gluing at their embranchment. One of terminal tube sections 3 and 4 is provided with a female connector 6, preferably a Luer connector.

In the lumen of the tonometric device (probe) of the invention there is a medium as a test material tending to achieve an equilibrium with the CO₂ content of the ambient along the whole length of the part of the device being inside the oral cavity. To display the test result, both a capnometric device provided with a pH-meter and a colometric measuring means are equally appropriate.

Measuring possibilities and measurements to be carried out using the tonometric device 1 of the invention are schematically discussed with reference to FIG. 2.

The balanced state of CO₂ content between the medium as test material inside the probe 1 and the outside environment occurs along the length of that part of probe 1 which is inside the oral cavity.

If air is used as test material the ambient air can also be used. In such a case, the probe 1 does not need to be loaded with air.

A connecting tube 8 is provided to transfer the test material from the probe 1 to the measuring unit 100. The connecting tube 8 is made of a material, which is less permeable for gases, such as polypropylene, and which has at an adequate wall thickness a low permeability for gases. For example, the connecting tube 8 has a length of 50 cm, an outer diameter of 2 mm and an inner diameter of 0.5 mm. For this purpose any polymers such as polytetrafluoro-ethylene (Teflon) or even softened polyvinylchloride (Tygon) can be used which have low permeability for gases.

If the test material is air, the measuring unit 100 is preferably a capnometer, which is generally available in medical institutions and detects the partial pressure of carbon dioxide (pCO₂) contained in the air transferred into its cell. When the test material is transferred from the probe 1 to the capnometer, in case of too fast displacement the test material can be mixed with the introduced streaming air (or other medium), whereby the result may become non-esteemable. Hence the air must be streamed slower, at a speed of 40-60 ml/minute. Such device is e.g. Sidestream Microcap Handheld Capnograph, Oridion Medical Ltd, Jerusalem, Israel.

The probe 1 can be loaded with filling liquid, too. The composition of the recommended solution as a filling liquid is as follows: 150 mM common salt (NaCl), 25 mM sodium hydrogen carbonate (NaHCO₃), and optionally a 1.5% by weight phenolic red. The phenolic red component is required only for detecting the pH change by the discolouration of the solution. The sodium-chloride and sodium-hydrogen-carbonate content of this filling liquid corresponds essentially to similar data of the plasma and other body fluids.

The measuring time takes about 10 minutes if a probe having an outer diameter of 2 mm and a wall thickness of 0.25 mm in case of air as test sample and about 15 minutes in case of liquid filling. After the lapse of the measuring time the test material formed from the filling liquid being in equilibrium with the CO₂ content of the environment can be transferred to the measuring unit by a medical syringe. For this purpose, the connecting stud of the syringe is inserted into the female connector 6 on terminal 4 of the probe 1. Then the test material is drawn through the connecting tube 8 by the syringe and is transferred e.g. to a pH-meter 110 such that it should fill up the measuring cells 110a or 110b of the pH-meter unit 110. Since the sodium hydrogen carbonate content of the test material is constant, the partial pressure of CO₂ in the solution can be determined from the pH value. In a state of equilibrium the relationship between the pH value of the filler liquid and the partial pressure (pCO₂) pertaining to the concentration of carbon dioxide are well known by a person skilled in the art, thus we do not touch upon the determination of the pCO₂ value. The partial pressure pertaining to the carbon dioxide concentration of the test material obtained from the probe 1 is determined by using the measuring unit 100.

The pH value of the filling liquid transferred from the probe 1 into the measuring unit 110 can be determined by conventional combined pH-meter electrodes (e.g. in the measuring cell 110a). It is important to keep the CO₂ content of the obtained test material at a constant level, therefore the sensor of the electrode is preferably placed into an air-proof measuring cell 110a or 110b provided with an inlet and an outlet. In case of a small volume of filling liquid, it is a preferred solution for determining of pCO₂ of the test sample to transfer the test material into a blood gas analysing apparatus commonly used in medical institutes in a manner that the sample is gathered in a glass capillary traded under the name “clinitube” (Radiometer, Copenhagen, Denmark) then analysed as usual.

The tonometric device according to the invention can be used for examining children older than 7 or 8 years of age. The single-use tonometric device stored in an appropriate sterile package is inserted into the mouth of the patient sublingually (FIG. 3a) or postlabially (FIG. 3b), as shown in the relevant FIGS. 3a and 3b. The tonometric device inserted into the mouth should be covered along its full extension by the lower surface of the tongue or one of the lips. Both the area under the tongue (FIG. 3a) and the area behind the lower or upper lips (FIG. 3b) are equally usable to locate the probe. During the examination, the patient or a medical assistant hold the connecting tube of the tonometric device between his/her forefinger and middle finger while he/she places his/her palm onto the lips without any pressure or strain and holds its position. In case of examination by using air, the tonometric device does not need advance filling. In case of examination by using a liquid, it is proceeded as above. Thereafter, with the examination expired, the connection tube of the measuring instrument is inserted into the Luer connector of the tonometric device and the measurement is carried out by using one of the procedures already described.

The determination of the pH value can be performed by examining the discolouration of the filling liquid containing phenolic red indicator. The phenolic red is a conventionally used and well-proved reagent for determining the pH value of solutions. The use of it in this solution is preferred since it is not toxic, on the one hand, and the transition point of the indicator is in the pH range from 7 to 8, on the other hand, which is essential in this case. After elapsing of the prescribed measuring time, the pH value of the filling liquid, which is exhausted air-proof from probe 1, can be determined with a photometer supplied with a through-pass type measuring cell 111 (cuvette). However, it should be noted that the because of the small volume of the tonometric device, it is feasible to use an apparatus is provided with a microcuvette for this purpose. For using different photometers, calibration is needed only one occasion. It is feasible to carry out the calibration by using an appropriate set of puffer solutions comprising 1.5 to 15 mg/l phenolic red.

In emergency cases based on the colour change of the indicator the tissue pCO₂ value can be quickly estimated. In extraordinary situations when no. measuring unit is available for the tonometric examination (for example in ambulance car), the pCO₂ value can be estimated by comparing the test material drawn from the probe 1 with a colour scale. The colour scale comprises five pieces of sealed test tubes having an internal diameter of 5 mm and comprising standard puffer solutions with pH value between 7.00 and 7.40 increasing by pH 0.1 each and comprising the same concentration of phenolic red as the test liquid. The standard tubes are labelled with the pCO₂ value pertaining to the pH value of the reference liquid. For the examination of the measuring liquid, a 1-ml syringe is inserted into the Luer connector of the device, and the other connecting side is placed into an empty glass test tube having an inside diameter of 5 mm then the test liquid is transferred into the test tube by using the syringe and the comparison is effected. The colour of the test fluid, except the upper 2 to 3 mm part, remains stable for approximately 10 minutes. Thereby the pCO₂ value of the sample can be estimated with an accuracy of 15 Hgmm. To carry out examination by using the probe 1 according to the invention, the patient does not need any preparation.

The expedient use of the probe 1 representing the tonometric device according to the invention is explained below.

The tonometric device stored in an appropriate sterile package and having an appropriate size is taken out from the package and is inserted into the mouth of the patient sublingually or postlabially. One end of the connecting tube is inserted into the female connector provided on the terminal of probe 1 provided with a Luer head, while the other end of it is connected to the measuring unit 100. For testing using air, the probe 1 does not need to be filled in advance. In case of testing using a test liquid, the test material is drawn through the female Luer connector and transferred to the interior of probe 1 in a way that the test liquid completely fills out the lumen of probe 1 while care is taken to avoid the testing liquid getting into the connecting tube 8. Thereafter, the lapse of a prescribed measurement period (1 to 10 minutes) is awaited. After the lapse of this period, the test material inside the probe 1 completely assumes the pCO₂ value of the surrounding mucous membrane. After the lapse of the test period, the test material is transferred from the probe 1 into the measuring unit 100 as already explained, and the result is read. The examination can be repeated at intervals as needed and interposing pauses expediently one-hour each.

As mentioned above, the invention can be used for controlling the systemic failure of the partial pressure belonging to the carbon-dioxide concentration of the body only with patients who are awake and capable of cooperation at least to some extent. Nevertheless, the examination can be carried out on patients who are unconscious or even in narcosis with the exception of intubated patients.

A significant advantage of the present invention is the easier, faster and more tolerable detection of the tissue perfusion failure of the body than in case of prior art. It should be emphasized that in practice, it can be used very easily without significant burdening of the patients especially due to the fact that the probe 1 can be made of a soft and flexible material and can be readily inserter into the mouth cavity. A further significant advantage lies in the fact that the specific surface of probe 1 is essentially exceeds that of known probes, allowing faster measurement. Said probe 1 can be used in emergency situations where of course only approximate results can be expected that serve only for orientation. Another advantage of the examination probes that their production is substantially cheaper than that of other means used for similar purposes and the probe can be repeatedly used if applied to the same patient after external rinsing with water.

The scope of the invention covers other embodiments and uses of the device according to the invention which differ from the instant disclosure but in the knowledge of the invention, are obvious for a person skilled in the art.

The device according to the invention is explained by the aid of the examples below. However, it should be noted that that any departure from the solutions discussed in the description and the examples does not represent an evasion of the scope of the invention if implemented in the spirit of the invention.

### EXAMPLE 1

A silicon rubber tube having an outer diameter of 3 mm and a wall thickness of 0.3 mm is folded according to FIG. 1a and is glued at locations marked by arrows c to g by an adhesive suitable for gluing silicon rubber. A polyurethane tube having an outer diameter of 2.5 mm and a wall thickness of 0.2 mm is inserted into both ends of the device prepared this way at points A and B as shown in FIG. 1a. To the end of one of the polyurethane tubes, a Luer connector is fixed.

The total width of rounded V-shaped sections laid alongside each other amounts to 18 mm if measured in the plane of the device. The distance between the ends of the rounded V-shaped sections amounts to 58 mm. The height of the rounded V-shape is 20 mm.

The device obtained this way has a volume of 1.4 ml. This device can be used for the examination of adult patients.

### EXAMPLE 2

It is proceeded fully as in case of Example 1 but the diameter of the applied silicon rubber tube is 2.5 mm and has a wall thickness of 0.25 mm. A further difference is that the silicon rubber tube is folded as shown in FIG. 1b and the terminals are formed from both ends of the tube, that is no separate polyurethane terminals are needed.

The total width of rounded V-shaped sections laid alongside each other amounts to 16 mm if measured in the plane of the device. The distance between the ends of the rounded V-shaped sections amounts to 54 mm. The height of the rounded V-shape is 19 mm.

The device obtained this way has a volume of 1.0 ml. This device can be used for the examination of infant patients.

1. A tonometric device (20) for examination of respiratory insufficiency and regional tissue perfusion failure in patients characterised in that said device (1) includes a silicone rubber tube (2) comprising adjacent rounded V shaped sections arranged alongside and connected to each other in a plane, a said device (1) including further a fibre (5) arranged inside said tube, said tube is provided with two terminals (3, 4), and said device (1) is adapted to be accommodated in sublingual or postlabial position in an oral cavity of the patient. (FIG. 1a)

2. The device of claim 1, characterised in that said sections of said silicone rubber tube are connected to each other by gluing.

3. The device of claim 1, characterised in that dimension a is 16 to 18 mm, dimension b is 52 to 62 mm, dimension h is 16 to 20 mm and said silicone rubber tube (2) has an outer diameter ranging from 1 to 3 mm, and a wall thickness less than 0.3 mm.

4. The device of claim 1 characterised in that said fibre (5) inside said tube (2) is a plastic fibre having a thickness ranging from 0.2 to 0.3 mm.

5. The device of claim 1, characterised in that one of said terminals is provided with a female connecting means (6).

6. The device of claim 1, characterised in that it comprises air or liquid as tilling material.

7. The device of claim 5, characterised in that as filling material it comprises a liquid including sodium chloride, sodium hydrogen carbonate and an indicator suitable for detecting carbon-dioxide concentration.

8. The device of claim 7, characterised in that it comprises phenolic red as indicator.

9. A kit for examination of respiratory insufficiency and regional tissue perfusion failure in patients, characterised in that it comprises a tonometric device (1) according to claim 1 and a measuring unit (100) that can be connected to said tonometric device (1) and is adapted for direct or indirect measurement of carbon dioxide.

10. A tonometric device (1) for examination of respiratory insufficiency and regional tissue perfusion failure in a patient, said device (1) including a silicone rubber tube (2) comprising adjacent rounded V shaped sections arranged alongside and connected to each other in a plane, said device (1) including further a fibre (5) arranged inside said tube, said tube is provided with two terminals (3, 4), and said device (1) is adapted to be accommodated in sublingual or postlabial position in an oral cavity of the patient.

11. The device of claim 10, characterised in that the total width of rounded V shaped sections as measured in the plane of the device is 16 to 18 mm, the distance between ends of rounded V shaped sections is 52 to 62 mm, the height of the rounded V shape is 16 to 20 mm and said silicone rubber tube (2) has an outer diameter ranging from 1 to 3 mm, and a wall thickness less than 0.3 mm.

12. The device of claim 10, characterised in that said fibre (5) inside said tube (2) is a plastic fibre having a thickness ranging from 0.2 to 0.3 mm and one of said terminals is provided with a female connector (6).

13. The device of claim 11, characterised in that one of said terminals is provided with a female connector (6).

14. A method for examination of respiratory insufficiency and regional tissue perfusion failure in patients, said method comprising directly or indirectly measuring carbon dioxide with a measuring unit (100) connected to the tonometric device (1) of claim 10.
